(19) United States
(12) Patent Application Publication
(10) Pub. No.: US 2018/0036501 A1
(43) Pub. Date: Feb. 8, 2018

(54) NASOGASTRIC TUBE

(71) Applicants: YU-JUI LIU, Nantou (TW); SHUN-CHUN LO, Taipei (TW); SHIAN-CHIUAN TZENG, Yunlin (TW); CHUN-LUNG LIN, Taipei (TW)

(72) Inventors: YU-JUI LIU, Nantou (TW); SHUN-CHUN LO, Taipei (TW); SHIAN-CHIUAN TZENG, Yunlin (TW); CHUN-LUNG LIN, Taipei (TW)

(21) Appl. No.: 15/666,490

(22) Filed: Aug. 1, 2017

Related U.S. Application Data
(60) Provisional application No. 62/372,278, filed on Aug. 8, 2016.

(30) Foreign Application Priority Data
Apr. 10, 2017 (TW) 106111910

(51) Int. Cl. A61M 16/04; A61J 15/00

(52) U.S. Cl. CPC A61M 16/0461; A61J 15/0003; A61M 25/0662

(57) ABSTRACT

The present disclosure discloses a nasogastric tube used to insert into a patient's body from nose to stomach. The patient's body from nose to stomach comprises a posterior pharyngeal wall, an inferior conch, a nasopharynx rear-upper wall, and a soft palate; the nasogastric tube comprises a tube body and a pressurized structure. The tube body has a longitudinal direction and includes a proximal end and a distal end. A distance is between and the posterior pharyngeal wall. The pressurized structure is disposed on the tube body along the longitudinal direction and near the distal end. The pressurized structure comprises a cavity. When a fluid is filled into the cavity, the pressurized structure is pressurized; and the distal end is lifted to reduce the distance to the posterior pharyngeal wall.

### CROSS-REFERENCE TO RELATED APPLICATIONS

This application claims priority to U.S. Provisional Patent Application No. 62/372,278, filed on Aug. 8, 2016, and claims priority to Taiwanese Patent Application No. 106111910 filed on Apr. 10, 2017, the contents of which are incorporated by reference herein.

### FIELD

The present disclosure relates to a nasogastric tube and, in particular, to a nasogastric tube having a pressurized structure.

### BACKGROUND

When a patient has difficulty in swallowing or has a problem in eating due to reasons such as coma, mouth and neck diseases, severe burns, cancer or surgery, a suggested treatment is to place a nasogastric tube from the nose to the stomach of the patient to force-feed food or drugs to the patient.

FIG. 1A is a schematic diagram showing a conventional nasogastric tube, and FIG. 1B is a schematic diagram showing the nasogastric tube of FIG. 1A inserted in the body of a patient P. In FIG. 1A, the nasogastric tube 4 includes a hollow tube 43 and a connector 42, which is disposed at a proximal end 41 of the hollow tube 43. In an insertion end of the nasogastric tube 4, a distal end 44 of the hollow tube 43 is inserted through the nose of the patient P. After the distal end 44 of the hollow tube 43 reaches the throat, the patient P has to do a swallowing action or drink some water to allow the hollow tube 43 to pass through the throat and the esophagus to reach the stomach. However, the hollow tube 43 is a flexible material, and the esophagus A4 is narrower than the trachea A5. It is hard to correctly insert the hollow tube 43 into the esophagus A4 when the distal end 44 of the hollow tube 43 reaches the epiglottis, which is above the esophagus A4. In some cases, the nasogastric tube 4 may be inserted into the mouth O or the trachea A5, which will cause coughing or aspiration pneumonia to the patient P. Even experienced medical personnel will need to try several times to properly complete the insertion of the nasogastric tube 4. For more difficult cases, it is necessary to use supplemental equipment, such as Magill intubating forceps, to insert the nasogastric tube 4 into the stomach of the patient P. However, the use of such equipment will cause discomfort to the patient and will need more time and cost to complete the procedure.

Therefore, it is desirable to provide a novel nasogastric tube that can help complete the insertion procedure in a more precise and easy manner, thereby reducing discomfort in the patient and saving precious medical cost.

### SUMMARY

In view of the foregoing, an objective of the present disclosure is to provide a nasogastric tube having a pressurized structure disposed near the distal end of the tube body. When a fluid (e.g. gas, liquid or semi-liquid) is filled into a cavity of the pressurized structure. The pressure of the tube body increases; and therefore the distal end of the tube body is drawn close to or in contact with the posterior pharyngeal wall, thereby avoiding the distal end from entering the mouth or trachea of the patient.

To achieve the above objective, the present disclosure discloses a nasogastric tube, which is used to inserted into a patient's body from nose to stomach. The body from nose to stomach comprises a posterior pharyngeal wall, an inferior conch, a nasopharynx rear-upper wall, and a soft palate. The nasogastric tube comprises a tube body and a pressurized structure. The tube body includes a proximal end and a distal end. A distance is between the distal end and the posterior pharyngeal wall. The pressurized structure is disposed on the tube body along the longitudinal direction and near the distal end. The pressurized structure has a cavity. When a fluid is filled into the cavity, the pressurized structure is pressurized, and a pressure of the tube body where the pressurized structure is disposed increases; and the distal end is lifted to reduce the distance to the posterior pharyngeal wall.

### BRIEF DESCRIPTION OF THE DRAWINGS

Implementations of the present disclosure will now be described, by way of example only, with reference to the attached figures.

FIG. 1A is a schematic diagram showing a conventional nasogastric tube.

FIG. 1B is a schematic diagram showing the nasogastric tube of FIG. 1A, which is inserted into a patient P.

FIG. 2A is a schematic diagram showing a nasogastric tube of the present disclosure according to a first preferred exemplary embodiment.

FIG. 2B is a cross-sectional view of the nasogastric tube of FIG. 2A along a longitudinal direction.

FIG. 2C is a cross-sectional view of the nasogastric tube of FIG. 2B along line L1-L1.

FIG. 2D is a cross-sectional view of the nasogastric tube of FIG. 2B along line L2-L2.

FIG. 2E is a cross-sectional view of the nasogastric tube of FIG. 2B along line L3-L3.

FIG. 2F is a cross-sectional view of the nasogastric tube of FIG. 2B along line L4-L4.

FIG. 2G is a schematic diagram showing the nasogastric tube of FIG. 2B; where a pressurized structure of the nasogastric tube is pressurized.

FIG. 3A is a cross-sectional view along a longitudinal direction of a nasogastric tube of the present disclosure according to a second preferred exemplary embodiment.

FIG. 3B a cross-sectional view of the nasogastric tube of FIG. 3A along line L5-L5.

FIG. 3C a cross-sectional view of the nasogastric tube of FIG. 3A along line L6-L6.

FIG. 3D a cross-sectional view of the nasogastric tube of FIG. 3A along line L7-L7.

FIG. 4A is a schematic diagram showing the nasogastric tube of FIG. 2A, which is inserted into a patient P, and where the pressurized structure of the nasogastric tube is not pressurized.

FIG. 4B is a schematic diagram showing the nasogastric tube of FIG. 2A, which is inserted into a patient, and where the pressurized structure of the nasogastric tube is pressurized.

FIG. 5A is a cross-sectional view along a longitudinal direction of a nasogastric tube of the present disclosure according to a third preferred exemplary embodiment.

FIG. 5B is a cross-sectional view of the nasogastric tube of FIG. 5A along line L8-L8.

FIG. 5C is a cross-sectional view of the nasogastric tube of FIG. 5A along line L9-L9.

FIG. 5D is a cross-sectional view of the nasogastric tube of FIG. 5A along line L10-L10.

FIG. 5E is a cross-sectional view of the nasogastric tube of FIG. 5A along line L11-L11.

FIG. 6A is a cross-sectional view along a longitudinal direction of a nasogastric tube of the present disclosure according to a fourth preferred exemplary embodiment.

FIG. 6B is a cross-sectional view of the nasogastric tube of FIG. 6A along line L12-L12.

FIG. 6C is a cross-sectional view of the nasogastric tube of FIG. 6A along line L13-L13.

FIG. 6D is a cross-sectional view of the nasogastric tube of FIG. 6A along line L14-L14.

FIG. 7 is a flow chart of a method of preparing the nasogastric tube of the present disclosure.

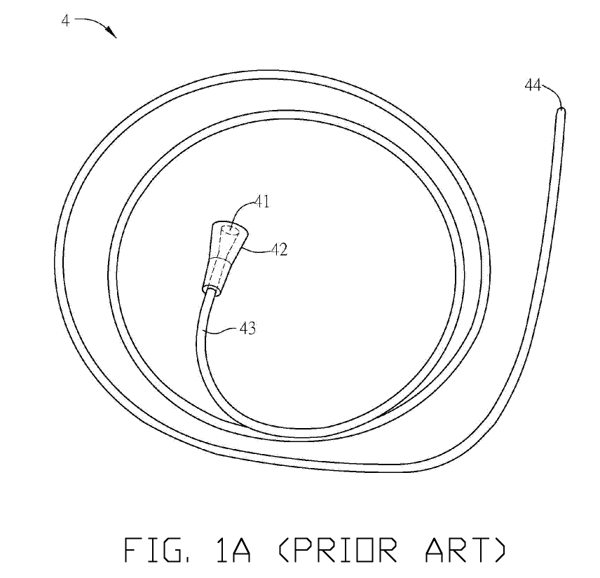

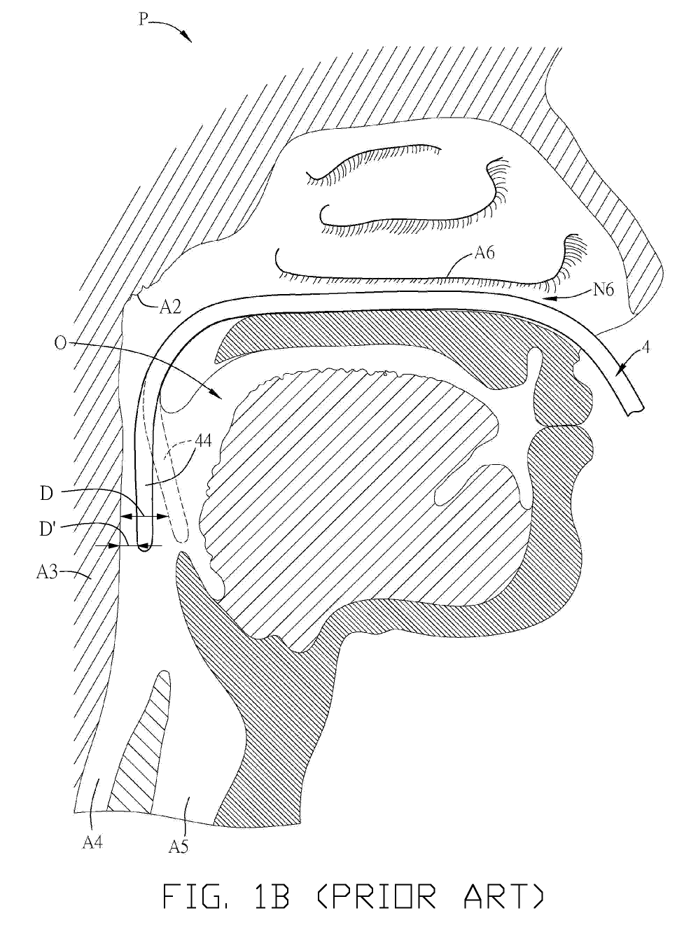

### DETAILED DESCRIPTION

The present disclosure will now be described more fully hereinafter with reference to the accompanying drawings, in which exemplary embodiments of the disclosure are shown. This disclosure may, however, be embodied in many different forms and should not be construed as limited to the exemplary embodiments set forth herein. Rather, these exemplary embodiments are provided so that this disclosure will be thorough and complete, and will fully convey the scope of the disclosure to those skilled in the art. Like reference numerals refer to like elements throughout.

The terminology used herein is for the purpose of describing particular exemplary embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

It will be understood that the term “and/or” includes any and all combinations of one or more of the associated listed items. It will also be understood that, although the terms first, second, third etc. may be used herein to describe various elements, components, regions, parts and/or sections, these elements, components, regions, parts and/or sections should not be limited by these terms. These terms are only used to distinguish one element, component, region, part or section from another element, component, region, layer or section. Thus, a first element, component, region, part or section discussed below could be termed a second element, component, region, layer or section without departing from the teachings of the present disclosure.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

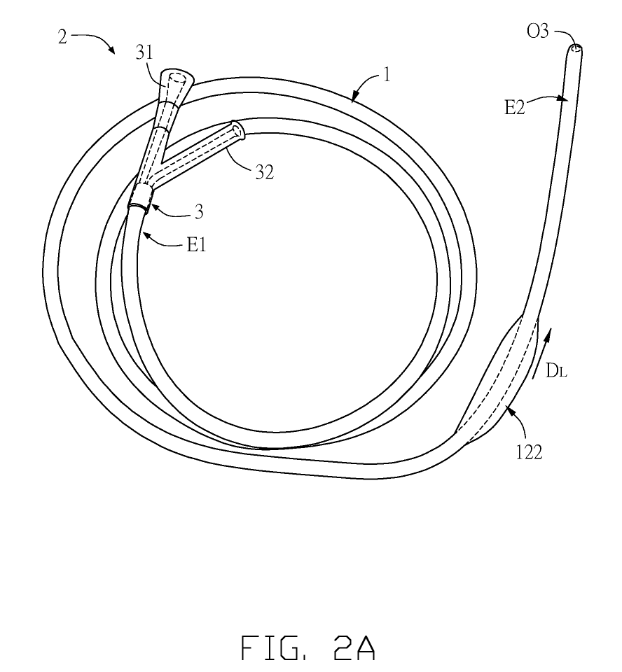

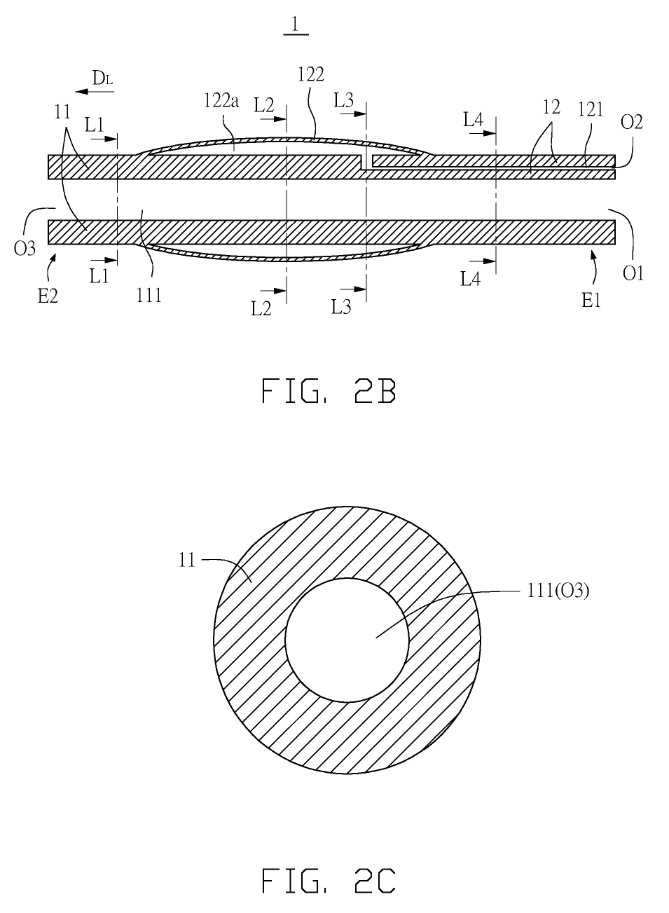

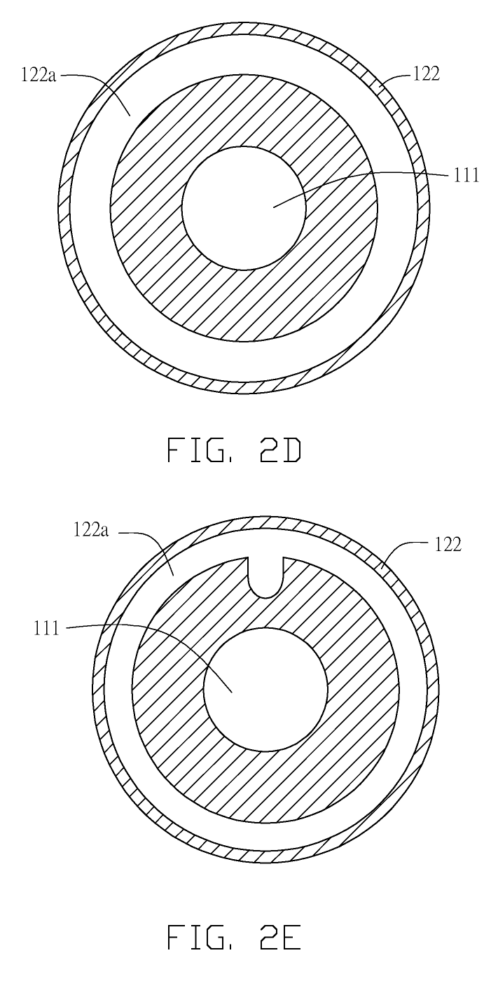

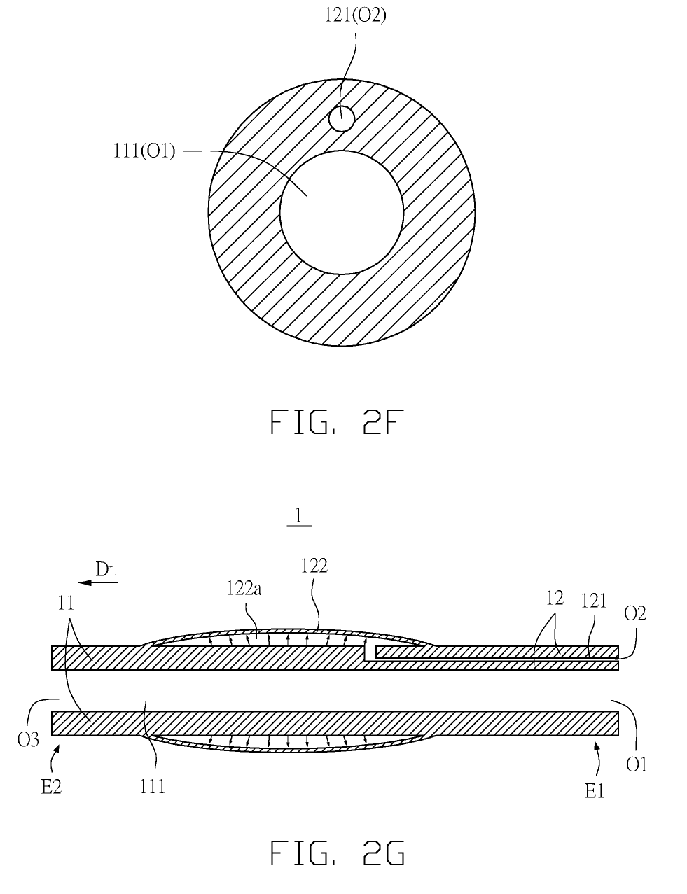

The description will be made as to the exemplary embodiments of the present disclosure in conjunction with the accompanying drawings in FIG. 2A to 7. Reference will be made to the drawing figures to describe the present disclosure in detail, wherein depicted elements are not necessarily shown to scale and wherein like or similar elements are designated by same or similar reference numeral through the several views and same or similar terminology.

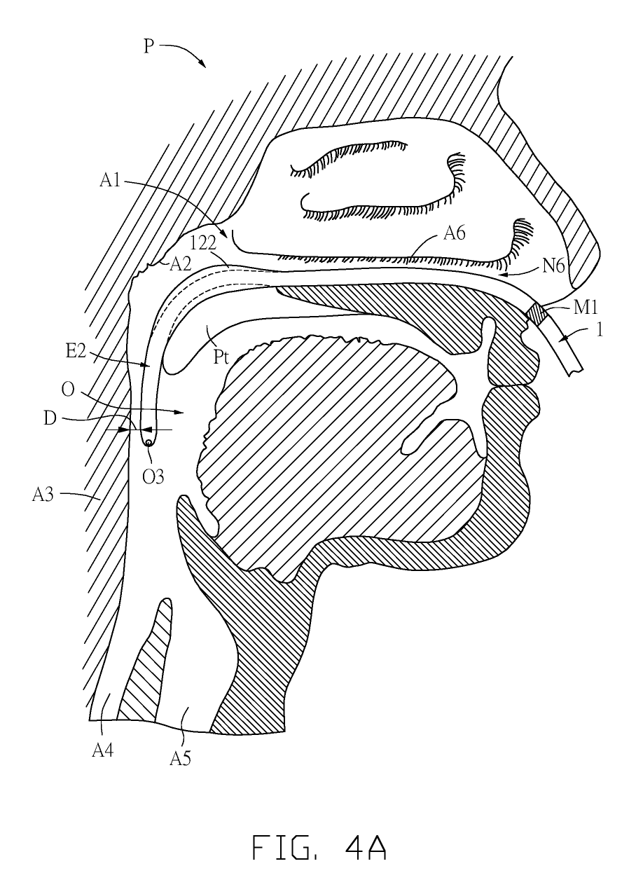

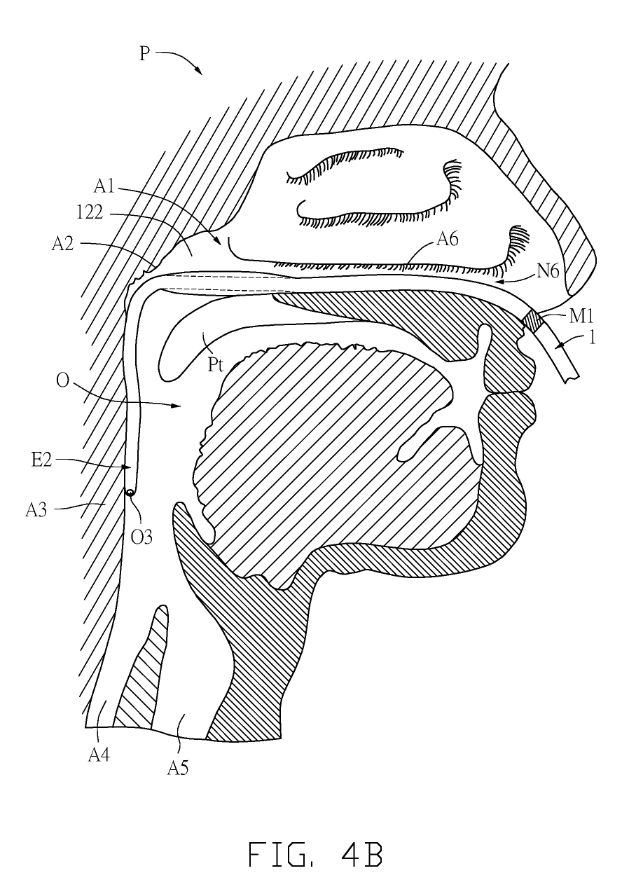

Referring to FIG. 2A to 4B, FIG. 2A is a schematic diagram showing a nasogastric tube of the present disclosure according to a first preferred exemplary embodiment. FIG. 2B is a cross-sectional view of the nasogastric tube of FIG. 2A along a longitudinal direction D_{L}. FIG. 2C is a cross-sectional view of the nasogastric tube of FIG. 2B along line L1-L1. FIG. 2D is a cross-sectional view of the nasogastric tube of FIG. 2B along line L2-L2. FIG. 2E is a cross-sectional view of the nasogastric tube of FIG. 2B along line L3-L3. FIG. 2F is a cross-sectional view of the nasogastric tube of FIG. 2B along line L4-L4. FIG. 2G is a schematic diagram showing the nasogastric tube of FIG. 2B; and where a pressurized structure of the nasogastric tube is pressurized. FIG. 4A is a schematic diagram showing the nasogastric tube of FIG. 2A, which is inserted into a patient P; and where the pressurized structure of the nasogastric tube is not pressurized. FIG. 4B is a schematic diagram showing the nasogastric tube of FIG. 2A, which is inserted into a patient P; and where the pressurized structure of the nasogastric tube is pressurized.

As shown in FIG. 2A and 4A, a nasogastric tube 2 according to a first preferred embodiment of the present disclosure is used to insert into a patient P's body from nose to stomach. When the nasogastric tube 2 enters the nose of the patient P, it will pass through an inferior meatus N6, a pharynx (including a nasopharynx A1, an oropharynx, and a hypopharynx), an esophagus A4, and finally reach the stomach. As shown in FIG. 4A, the patient P's body from nose to stomach comprises a posterior pharyngeal wall A3, an inferior conch A6, a nasopharynx rear-upper wall A2, and a soft palate Pt. The nasopharynx rear-upper wall A2 indicates a rear-upper region of the nasopharynx A1, as the region marked with A2 of FIG. 4A and 4B. As shown in FIG. 2A, the nasogastric tube 2 of the present disclosure according to the first preferred embodiment comprises a tube body 1 and a pressurized structure 122. The tube body 1 haves a longitudinal directional D_{L}. The tube body 1 comprises a proximal end E1 and a distal end E2. The pressurized structure 122 is disposed on the tube body 1 along the longitudinal direction D_{L} and near the distal end E2. As shown in FIG. 4A, a distance D1 is between the distal end E2 and posterior pharyngeal wall A3.

The tube body 1 can be made of a material that is soft and flexible and is nonperishable, such as silica gel. After the nasogastric tube 2 enters the nose of the patient P, it will pass through the inferior meatus N6, the pharynx (including the nasopharynx A1, the oropharynx, and the hypopharynx), the esophagus A4, and finally reach the stomach. In practice, the proximal end E1 is an injection end of the nasogastric tube 2 for connecting the feeding syringe. In other words, the proximal end E1 can be used for feeding food or drawing the residuals in the stomach. The distal end E2 is a leading end of the nasogastric tube 2 for guiding the nasogastric tube 2 into the nose and the esophagus A4.

Referring to FIG. 2B, the tube body 1 further includes a first tube 11 and a second tube 12. The first tube 11 has a first tube chamber 111. The second tube 12 has a second tube chamber 121. The first tube 11 and the second tube 12 of the nasogastric tube 2 are in parallel along the longitudinal direction of the tube body 1. Accordingly, the nasogastric tube 2 has dual channels. As shown in FIG. 2B, the first tube 11 and the second tube 12 are preferably made of the same material and are integrally formed in one piece.

Referring to FIGS. 2B to 2G specifically, the tube body according to the first preferred exemplary embodiment further comprises a first tube 11 and a second tube 12. The first tube 11 comprises a first tube chamber 111. The second tube 12 is disposed adjacent to the first tube 11. The second tube 12 comprises a second tube chamber 121. The first tube 11 and the second tube 12 of the nasogastric tube 2 are disposed along the longitudinal direction D_{L} of the tube body 1. The first tube 11 and the second tube 12 form a double-lumen type of nasogastric tube. The first tube 11 and the second tube 12 can be integrally formed by a single material. Alternatively, the first tube 11 and the second tube 12 can be jointly made of different materials.

As shown in FIG. 2B, the proximal end E1 of the tube body 1 has two openings, including a first opening O1 and a second opening O2. Besides, as shown in FIG. 2A and 2B, the distal end E2 of the tube body 1 has a third opening O3. The first opening O1 is in air communication with the third opening O3 through the first tube chamber 111. That is, the first tube 11 is an open tube, and has the first opening O1 at the proximal end E1 and the third opening O3 at the distal end E2. The second tube chamber 121 is in air communication with the second opening O2; and the second tube chamber 121 is closed at the distal end E2. That is, the second tube 12 has an open end and a closed end. As shown in FIG. 2F, the first opening O1 at the proximal end E1 of the tube body 1 is the first tube chamber 111, and the second opening O2 at the proximal end E1 is the second tube chamber 121. Preferably, the first tube chamber 111 has a diameter of about 0.2 to 0.4 centimeter; and the second tube chamber 121 has a diameter of about 0.O3 to 0.1 centimeter. In this exemplary embodiment, the first tube 11 and the second tube 12 is integrally formed by a single material. Alternatively, the first tube 11 and the second tube 12 can be jointly made of different materials. The first tube chamber 111 and the second tube chamber 121 are isolated from each other and can be used in different purposes.

Referring to FIG. 2A to 2G the pressurized structure 122 is disposed on the tube body 1 and located between the proximal end E1 and the distal end E2. The pressurized structure 122 is near the distal end E2. In this exemplary embodiment, the pressurized structure 122 is a cylinder structure sleeved on the first tube 11 and concentric with the first tube 11. A distance between the pressurized structure 122 and the distal end E2 is about 1 to 9 centimeters. Preferably, the distance between the pressurized structure 122 and the distal end E2 is about 5 to 9 centimeters. The pressurized structure 122 has a length of about 1 to 10 centimeters along the longitudinal direction D_{L}. Preferably, the pressurized structure 122 has a length of about 4 to 8 centimeters along the longitudinal direction D_{L}. The pressurized structure 122 further comprises a cavity 122a. The cavity 122a has a height, perpendicular to the longitudinal direction D_{L}, of about 0.2 to 1.2 centimeters. The cavity 122a is in air communication with each other and the second tube chamber 121 through an opening of the side wall of the second tube 12. The cavity 122a can be filled with a fluid (including a gas, a liquid, or a semiliquid), and hence the pressurized structure 122 is pressurized. Therefore, the pressure of the tube body 1, allows for the tube body 1, where the pressurized structure 122 is disposed, to be higher than other parts of the tube body 1.

When a medical personnel is preparing the nasogastric tube 2, the pressurized structure 122 can be filled with the fluid to be pressurized, and then be inserted into a patient's body. Alternatively, in order to reduce the foreign body sensation of the patient, the pressurized structure 122 can be unpressurized at the beginning of the insertion process. In other words, the pressurized structure 122 is unpressurized before reaching a posterior end of the inferior conch A6 or passing through the inferior conch A6. Meanwhile, the cavity 122a is not yet filled with the fluid, as shown in FIG. 4A. When the pressurized structure 122 reaches the posterior end of the inferior conch A6 or passes through the inferior conch A6, the pressurized structure 122 is filled with the fluid to be pressurized, as shown in FIG. 4B.

As shown in FIG. 4B, the fluid filled into the pressurized structure 122 is to increase the pressure of the tube body 1 where the pressurized structure 122 is disposed. In such a way, compared to other parts of the tube body 1, the tube body 1 where the pressurized structure 122 is disposed becomes stiff due to an increase in pressure. In other words, compared to other parts of the tube body 1, a curvature of the tube body 1 where the pressurized structure 122 is disposed reduces. When the curvature of the tube body 1 where the pressurized structure 122 is disposed reduces, the distal end E2 is lifted to reduce the distance D1 to the posterior pharyngeal wall A3 or even to be in contact with the posterior pharyngeal wall A3, as shown in FIG. 4B. Therefore, the distal end E2 can be guided into the esophagus A4. The fluid can be a gas, a liquid, or a semiliquid, such as normal saline, gel, lotion, or a mixture of solid and liquid.

As shown in FIG. 2A, the nasogastric tube 2 may further comprises a Y connector 3. The Y connector 3 is disposed on the proximal end E1 of the tube body 1. The Y connector 3 comprises a first connecting port 31 and a second connecting port 32. The first connecting port 31 is in air communication with the first tube chamber 111 through the first opening O1. The second connecting port 32 is in air communication with the second tube chamber 121 through the second opening O2.

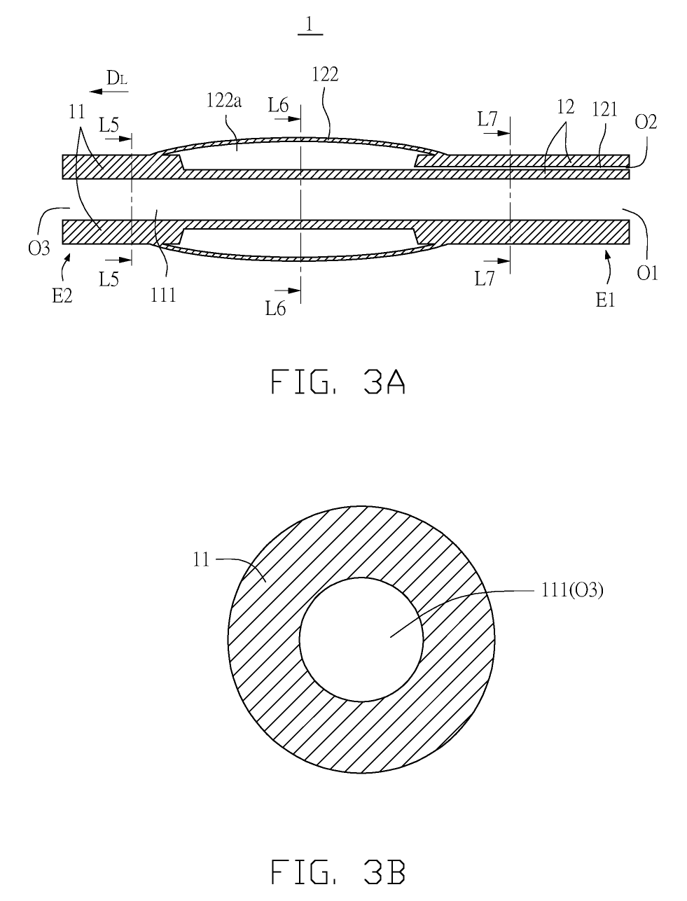

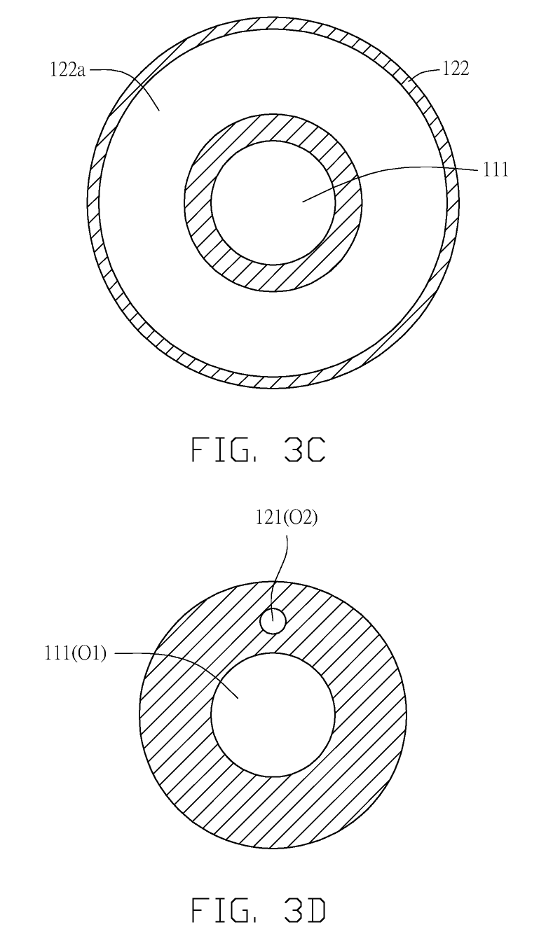

Referring to FIG. 3A to 3D, FIG. 3A is a cross-sectional view along a longitudinal direction of a nasogastric tube of the present disclosure according to a second preferred exemplary embodiment. FIG. 3B a cross-sectional view of the nasogastric tube of FIG. 3A along line L5-L5. FIG. 3C a cross-sectional view of the nasogastric tube of FIG. 3A along line L6-L6. FIG. 3D a cross-sectional view of the nasogastric tube of FIG. 3A along line L7-L7. The structure of the nasogastric tube 2 and the pressurized structure 122 of FIG. 3A is similar to that of FIG. 2B; and the pressurized structure 122 in FIG. 3A is also a cylinder structure sleeved on the first tube 1. The difference between the nasogastric tube 2 of FIG. 3A and FIG. 2B is that: as shown in FIG. 3A and 3C, the tube body 1 where the pressurized structure 122 is disposed has a thickness less than that of other parts of the tube body 1. In other words, compared to the first tube 11 shown in the cross-sectional view of FIG. 3B, the first tube 11 shown in the cross-sectional view of FIG. 3C has a smaller thickness. In such a way, the pressurized structure 122 and the nasogastric tube 2 of this exemplary embodiment can achieve a similar purpose as described in the first preferred exemplary embodiment without further description.

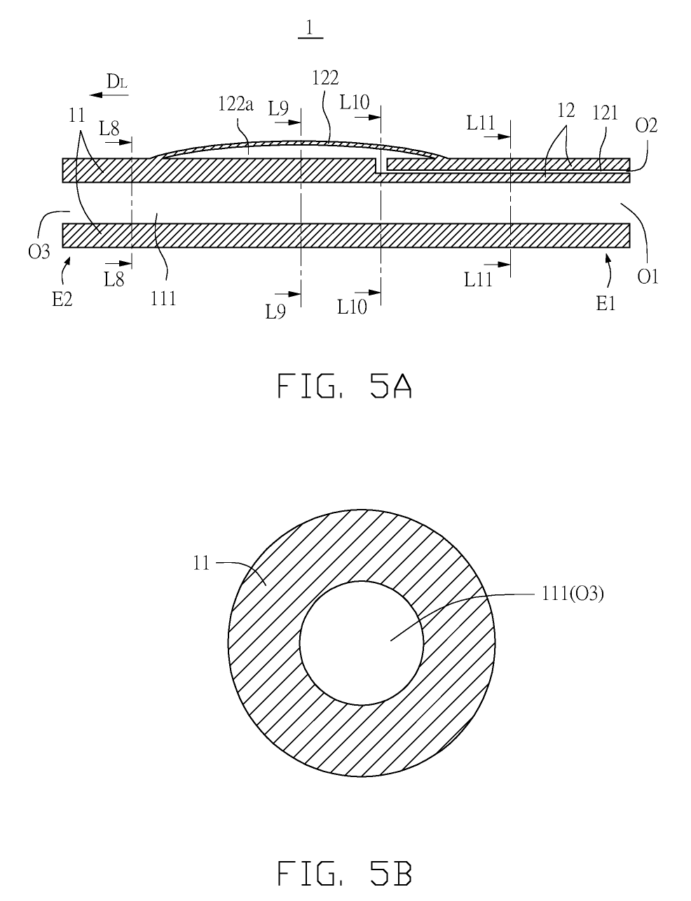

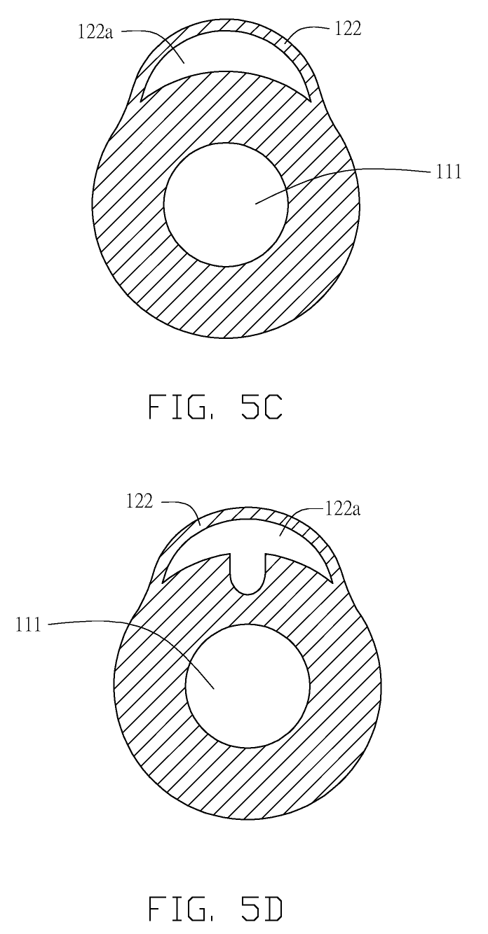

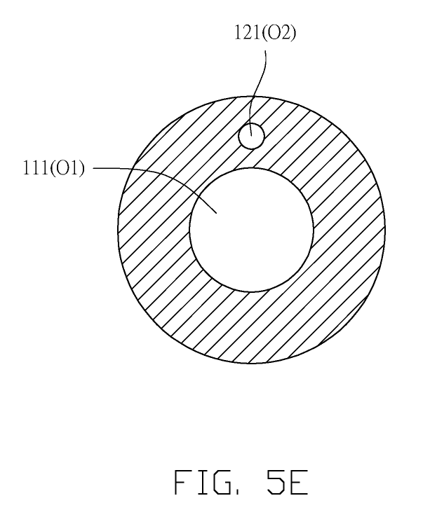

Referring to FIG. 5A to 5E, FIG. 5A is a cross-sectional view along a longitudinal direction of a nasogastric tube of the present disclosure according to a third preferred exemplary embodiment. FIG. 5B is a cross-sectional view of the nasogastric tube of FIG. 5A along line L8-L8. FIG. 5C is a cross-sectional view of the nasogastric tube of FIG. 5A along line L9-L9. FIG. 5D is a cross-sectional view of the nasogastric tube of FIG. 5A along line L10-L10. FIG. 5E is a cross-sectional view of the nasogastric tube of FIG. 5A along line L11-L11. As shown in FIG. 5A, 5C and 5D, the difference between the third preferred exemplary embodiment and the first and second preferred exemplary embodiments is that: instead of a cylinder structure sleeved on the first tube 11, in the third preferred exemplary, the pressurized structure 122 is disposed on one side of the first tube 11. In such a way, the pressurized structure 122 and the nasogastric tube 2 of this exemplary embodiment can achieve a similar purpose as described in the first and second preferred exemplary embodiments without further description.

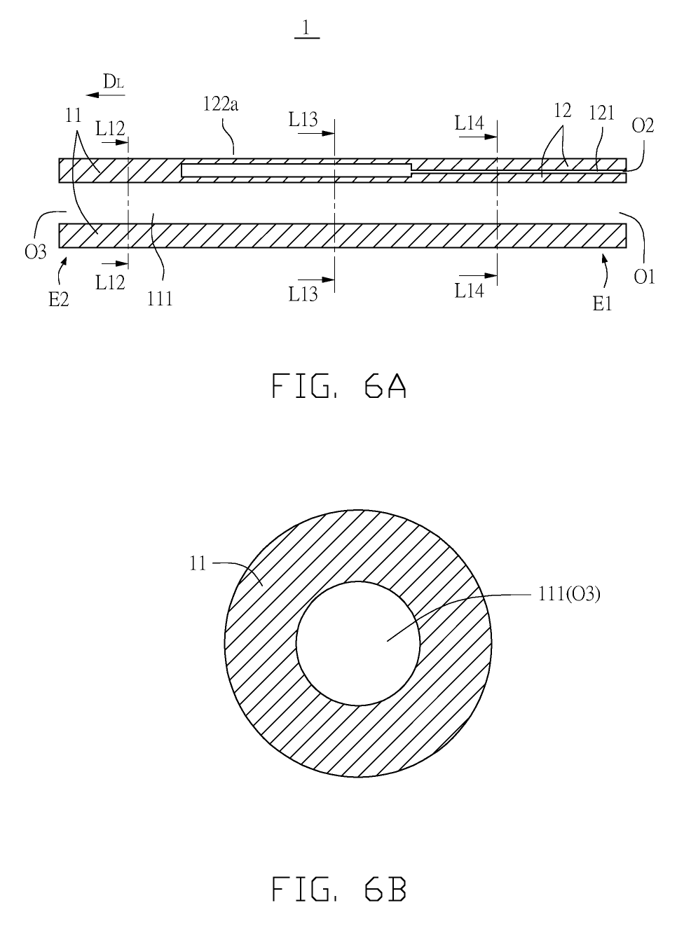

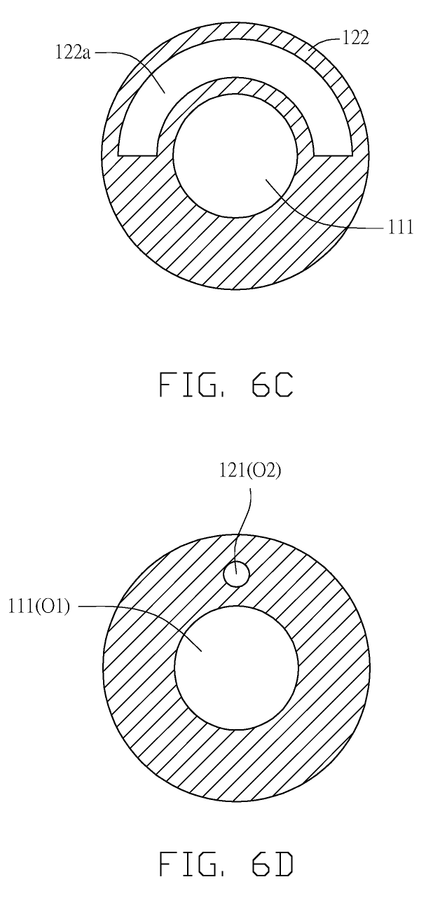

Referring to FIG. 6A to 6D, FIG. 6A is a cross-sectional view along a longitudinal direction of a nasogastric tube of the present disclosure according to a fourth preferred exemplary embodiment. FIG. 6B is a cross-sectional view of the nasogastric tube of FIG. 6A along line L12-L12. FIG. 6C is a cross-sectional view of the nasogastric tube of FIG. 6A along line L13-L13. FIG. 6D is a cross-sectional view of the nasogastric tube of FIG. 6A along line L14-L14. As shown in FIG. 6A and 6C, instead of a cylinder structure sleeved on the first tube 11, the pressurized structure 122 of this embodiment is disposed on one side of the first tube 11. Also, the pressurized structure 122 is formed within one side wall of the first tube 11; and the cavity 122a is a chamber within the side wall of the first tube 11. The pressurized structure 122 and the first tube 11 can be integrally formed by a single material. Alternatively, the pressurized structure 122 and the first tube 11 can be jointly made of different materials. As shown in FIG. 6A and 6C, a thickness of the tube body 1 where the pressurized structure 122 is disposed is smaller than that of other parts of the tube body 1. In such a way, the pressurized structure 122 and the nasogastric tube 2 of this exemplary embodiment can achieve a similar purpose as described in the previous exemplary embodiments without further description.

In practice, the medical personnel connects the feeding syringe (not shown) to the first connecting port 31 of the Y connector 3, and then injects liquid foods or drugs to the stomach of the patient through the first opening O1, the first tube chamber 111 and the third opening O3. Besides, the connecting port 32 is used to fill the fluid (e.g. gas, liquid, or semiliquid) into the cavity 122a of the pressurized structure 122, or to drain the fluid from the cavity 122a, as described in detail below. In practice, the medical personnel can discharge the fluid into the second tube chamber 121 from the second connecting port 32 of the Y connector 3. The fluid enters the second tube chamber 121 through the second opening O2, and then flows into the cavity 122a to pressurize the pressurized structure 122.

Before the insertion process of the nasogastric tube 2, the medical personnel can fill the fluid into the cavity 122a to pressurize the pressurized structure 122 from the second connecting port 32 of the Y connector 3 and through the second opening O2 and the second tube chamber 121. Alternatively, in order to reduce the foreign body sensation of the patient P, the pressurized structure 122 can be unpressurized at the beginning of the insertion process. In other words, the pressurized structure 122 is unpressurized before reaching a posterior end of the inferior conch A6 or passing through the inferior conch A6. Meanwhile, the cavity 122a is not yet filled with the fluid, as shown in FIG. 4A.

Referring to FIG. 2G and 4B, when the pressurized structure 122 reaches the patient P's posterior end of the inferior conch A6 or passes through the inferior conch A6, the medical personnel fills the fluid into the cavity 122a from the second connecting port 32 of the Y connector 3 and through the second opening O2 and the second tube chamber 121. Therefore, the pressurized structure 122 is pressurized, and becomes stiff. The tube body 1 where the pressurized structure 122 is disposed also becomes stiff. In other words, the curvature of the tube body 1 where the pressurized structure 122 is disposed reduces. As shown in FIG. 4B, the pressure of the tube body 1 where the pressurized structure 122 is disposed increases by filling the cavity 122a with the fluid; and the curvature of the tube body 1 between the nasopharynx rear-upper wall A2 and the soft palate Pt reduces; and therefore the distal end E2 is lifted to reduce the distance to the posterior pharyngeal wall A3 or even to be in contact with the posterior pharyngeal wall A3. Specifically, as shown in FIG. 4A, since the tube body 1 is made of soft material, when the pressurized structure 122 is not pressurized, the tube body 1 between the between the nasopharynx rear-upper wall A2 and the soft palate Pt is bent due to gravity. When the cavity 122a is filled with the fluid, the pressurized structure 122 is pressurized; and the tube body 1 where the pressurized structure 122 is disposed becomes stiff, as shown in FIG. 4B. Meanwhile, the distal end E2 is lifted to reduce the distance to the posterior pharyngeal wall A3 or even to be in contact with the posterior pharyngeal wall A3. In such a way, the distal end E2 can be guided into the esophagus A4 to avoid entering the trachea A5 or the mouth O.

Referring to FIG. 4A and 4B, the insertion of the nasogastric tube 2 is further illustrated. As shown in FIG. 4A, during the insertion process, the medical personnel inserts the distal end E2 of the nasogastric tube 2 into the nose of the patient P. Then, the distal end E2 passes through the inferior meatus N6 and then enters the nasopharynx A1. Next, the medical personnel can determine whether the pressurized structure 122 is close to the posterior end of the inferior conch A6 or has passed through the inferior conch A6 by the mark M disposed on the tube body 1. For example, the mark M is located at the nostril of the patient P. When the pressurized structure 122 is close to the posterior end of the inferior conch A6 or after the pressurized structure 122 passes through the inferior conch A6, the distal end E2 is located at the oropharynx that is above the esophagus A4. At this moment, the medical personnel can fill the fluid through the second connecting port 32 of the Y connector 3. The fluid flows through the second opening O2 and the second tube chamber 121, and enters the cavity 122a. Accordingly, the pressurized structure 122 is pressurized.

Furthermore, as shown in FIG. 4B, the medical personnel continues to fill the fluid into the cavity 122a to keep the pressurized structure 122 pressurized. Meanwhile, the medical personnel continues to insert the tube body 1, so that the pressurized structure 122 is pushed forward to the nasopharynx rear-upper wall A2. Accordingly, due to the longitudinal stress, the tube body 1 where the pressurized structure 122 is disposed becomes stiff; and therefore the distal end E2 is lifted to reduce the distance to the posterior pharyngeal wall A3 or even to be in contact with the posterior pharyngeal wall A3. Thus, the distal end E2 enters the esophagus A4 along the posterior pharyngeal wall A3. The configuration of the nasogastric tube 2 of the present disclosure can reduce the distance D1 between the distal end E2 of the tube body 1 and the posterior pharyngeal wall A3 or make the distal end E2 contact with posterior pharyngeal wall A3. In such a way, the possibility of the distal end E2 of the nasogastric tube 2 entering the esophagus A4 is increased; and hence the possibility of the distal end E2 entering the patient P's trachea A5 or mouth O is reduced.

When the distal end E2 gets close to the posterior pharyngeal wall A3 of the patient P and then successfully passes through the oropharynx or enters the esophagus A4, the medical personnel can continue to insert the nasogastric tube 2 to dispose the distal end E2 in the patient P's stomach. When the distal end E2 is disposed in the patient P's stomach, the medical personnel can drain the fluid from the second tube chamber 121 through the second connecting port 32 of the Y connector 3; and thereby the pressure of the pressurized structure 122 reduces. Then, the medical personnel can inject fluid foods or drugs into the stomach of the patient P through the first connecting port 31 of the Y connector 3, the first opening O1 and the first tube chamber 111. Alternatively, the medical personnel can draw residuals from the stomach of the patient P.

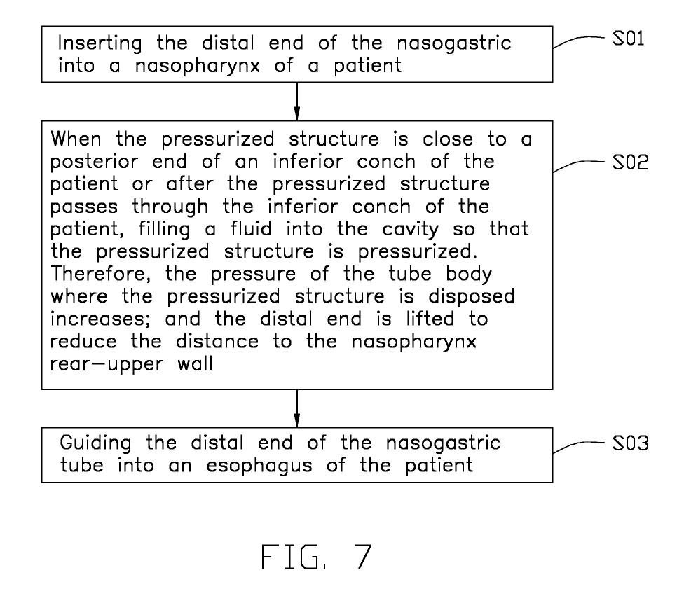

The present disclosure also discloses a method of preparing a nasogastric tube. FIG. 7 is a flow chart of the method of preparing the nasogastric tube according to an exemplary embodiment of the present disclosure. The nasogastric tube includes a tube body and a pressurized structure. The tube body has a longitudinal direction and includes a proximal end and a distal end. The pressurized structure is disposed on the tube body along the longitudinal direction and near the proximal end. The pressurized structure comprises a cavity. The structure of the nasogastric tube and the configuration of its components are described in the above exemplary embodiments without further description.

The method of preparing a nasogastric tube includes the following steps. In step S01, the distal end of the nasogastric tube is inserted into a nasopharynx of a patient. In step S02, when the pressurized structure is close to a posterior end of an inferior conch of the patient or after the pressurized structure passes through the inferior conch of the patient, a fluid is filled into the cavity so that the pressurized structure is pressurized. Therefore, the pressure of the tube body where the pressurized structure is disposed increases; and the distal end is lifted to reduce the distance to the nasopharynx rear-upper wall. In step S03, the distal end of the nasogastric tube can be guided into an esophagus of the patient. According to the above three steps of the method, the nasogastric tube can be properly placed in the stomach of the patient.

Moreover, after the step of guiding the distal end of the nasogastric tube into the esophagus of the patient, the method of preparing a nasogastric tube further includes a step of draining the fluid through the second tube chamber and the second connecting port so as to depressurize the pressurized structure.

The other technical features of the method of preparing a nasogastric tube can be referred to the above exemplary embodiments without further description.

In summary, the nasogastric tube of the present disclosure has a pressurized structure disposed on the tube body and near the distal end. The pressurized structure has a cavity. When a fluid is filled into the cavity, the pressurized structure is pressurized; and the pressure of the tube body where the pressurized structure is disposed increases. The distal end is lifted to reduce the distance to the nasopharynx rear-upper wall. Therefore, the distal end of the tube body is guided into the esophagus of the patient. Thus, the medical personnel can easily insert the nasogastric tube through the throat, past the esophagus and down to the stomach. This configuration can effectively decrease the failed insertion of the nasogastric tube. The nasogastric tube of the present disclosure can improve the controlling and confidence of the medical personnel for inserting the nasogastric tube, reduce the discomfort or fear of the patient in the nasogastric tube insertion, and save medical cost.

The exemplary embodiments shown and described above are only examples. Many details are often found in the art such as the other features of a nasogastric tube. Therefore, many such details are neither shown nor described. Even though numerous characteristics and advantages of the present technology have been set forth in the foregoing description, together with details of the structure and function of the present disclosure, the disclosure is illustrative only, and changes may be made in the detail, especially in matters of shape, size, and arrangement of the parts within the principles of the present disclosure, up to and including the full extent established by the broad general meaning of the terms used in the claims. It will therefore be appreciated that the exemplary embodiments described above may be modified within the scope of the claims.

What is claimed is:

1. A nasogastric tube, used to insert into a patient's body from nose to stomach; wherein the patient's body from nose to stomach comprises a posterior pharyngeal wall, an inferior conch, a nasopharynx rear-upper wall, and a soft palate; the nasogastric tube comprising:
a tube body with a longitudinal direction, having a proximal end and a distal end; wherein a distance is between the distal end and the posterior pharyngeal wall;
a pressurized structure disposed on the tube body along the longitudinal direction and near the distal end; and
wherein the pressurized structure comprises a cavity; when a fluid is filled into the cavity, the pressurized structure is pressurized, a pressure of the tube body where the pressurized structure is disposed increases, and the distal end is lifted to reduce the distance to the posterior pharyngeal wall.

2. The nasogastric tube of claim 1, wherein the fluid is a gas, a liquid, or a semi-liquid.

3. The nasogastric tube of claim 1, wherein the fluid filled into the cavity is to increase the pressure of the tube body where the pressurized structure is disposed, and thereby a curvature of the tube body where the pressurized structure is disposed reduces.

4. The nasogastric tube of claim 1, wherein a distance between the pressurized structure and the distal end is about 1 to 9 centimeters.

5. The nasogastric tube of claim 1, wherein the pressurized structure has a length of about 1 to 10 centimeters along the longitudinal direction.

6. The nasogastric tube of claim 1, wherein the proximal end comprises a first opening and a second opening; the distal end comprises a third opening; and the tube body further comprises:
a first tube comprising a first tube chamber; wherein the first opening is in air communication with the third opening through the first tube chamber; and
a second tube disposed along a longitudinal direction of the first tube; wherein the second tube comprises a second tube chamber; and the second opening is in air communication with the cavity of the pressurized structure through the second tube chamber.

7. The nasogastric tube of claim 6, wherein the pressurized structure is a cylinder structure sleeved on the first tube; and the pressurized structure is concentric with the first tube.

8. The nasogastric tube of claim 6, wherein the pressurized structure is disposed on one side of the first tube.

9. The nasogastric tube of claim 1, further comprising a Y connector; wherein the Y connector is disposed on the proximal end; the Y connector comprises a first connecting port and a second connecting port; the first connecting port is in air communication with the first tube chamber through the first opening; and the second connecting port is in air communication with the second tube chamber through the second opening.

10. The nasogastric tube of claim 6, wherein the second tube is sleeved on the first tube.

11. The nasogastric tube of claim 6, wherein the second tube is disposed adjacent to the first tube.
